(19) United States
(12) Patent Application Publication
(10) Pub. No.: US 2009/0198096 A1
(43) Pub. Date: Aug. 6, 2009

(54) LONG FATIGUE LIFE CARDIAC HARNESS

(75) Inventors: Peter Martin, Mountain View, CA (US); Matthew Fishler, Sunnyvale, CA (US); Tedd Hinton, San Jose, CA (US); Craig Mar, Fremont, CA (US); Anuja Patel, San Francisco, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: PARACOR MEDICAL, INC., Sunnyvale, CA (US)

(21) Appl. No.: 12/367,833

(22) Filed: Feb. 9, 2009

Related U.S. Application Data
(63) Continuation-in-part of application No. 12/212,013, filed on Sep. 17, 2008.
Application No. 12/212,013 is a division of application No. 10/694,646, filed on Oct. 27, 2003, now Pat. No. 7,455,738.

(51) Int. Cl. A61F 2/00

(52) U.S. Cl. 600/37

(57) ABSTRACT

A high fatigue life superelastic nickel-titanium (nitinol) wire, ribbon, sheet, tubing, or the like is disclosed. The nitinol has a 54.5 to 57.0 weight percent nickel with a balance of titanium composition and has less than 30 percent cold work as a final step after a full anneal and before shape setting heat treatment. Through a rotational beam fatigue test, fatigue life improvement of 37 percent has been observed.

### CROSS-REFERENCES TO RELATED APPLICATIONS

This application is a continuation-in-part of U.S. application Ser. No. 12/212,013, filed Sep. 17, 2008, which is a divisional of U.S. application Ser. No. 10/694,646 filed Oct. 27, 2003, and the entire contents of both disclosures are incorporated by reference and priority is claimed thereto.

### BACKGROUND

The present invention relates to a method and apparatus for providing a superelastic metal alloy having improved fatigue life. In particular, the present invention relates to a long fatigue life nickel-titanium alloy wire, ribbon, tubing, or sheet, a medical device such as a cardiac harness or stent made from the long fatigue life nickel-titanium alloy wire, ribbon, tubing, and sheet, and a method of using the medical device.

There has been great interest in shape memory and superelastic alloys such as nickel-titanium. This family of alloys, also known as nitinol (i.e., Nickel-Titanium Naval Ordinance Laboratory), is typically made from a roughly equal composition of nickel and titanium. A key to exploiting the performance of nitinol alloys is the phase transformation in the crystalline structure that transitions between an austenitic phase and a martensitic phase. The austenitic phase is commonly referred to as the high temperature phase, and the martensitic phase is commonly referred to as the low temperature phase. The back and forth phase changes is the mechanism for achieving superelasticity and the shape memory effect.

As the name implies, shape memory means that the alloy can be twisted into a particular shape in the martensitic phase, and then when heated to the austenitic phase, the metal returns to its remembered shape. In contrast, superelasticity refers to the ultra high elastic behavior of the alloy under stress. Typical reversible strains of up to 8 percent elongation can be achieved in a superelastic nitinol wire as compared to 0.5 percent reversible strain in a steel wire, for example. This superelasticity appears in the austenitic phase when stress is applied to the alloy and the alloy changes from the austenitic phase to the martensitic phase. This particular martensitic phase is more precisely described as stress-induced martensite (SIM), which is unstable at temperatures above A_{f} (the austenitic finish) temperature. As such, if the applied stress is removed, the stress-induced martensite reverts back to the austenitic phase. It is understood that this phase change is what enables the characteristic recoverable strains achievable in superelastic nitinol.

Nitinol was originally developed by the military, but has found its way into many commercial applications. Applications that utilize the shape memory effect of the alloy include pipe couplings, orthodontic wires, bone staples, etc. Products that exploit the superelasticity of nitinol include, for example, antennas and eye glass frames.

The medical device industry has also found many uses for nitinol. Nitinol has been used to fabricate guide wires, cardiac pacing leads, prosthetic implants such as stents, intraluminal filters, and tools deployed through a cannula, to name a few. Such devices are taught in, for example, U.S. Pat. Nos. 4,665,906; 5,067,957; 5,190,546; 5,597,378; 6,306,141; and 6,533,805 to Jervis; U.S. Pat. Nos. 5,486,183; 5,509,923; 5,632,746; 5,720,754; 5,749,879; 5,820,628; 5,904,690; 6,004,330; and 6,447,523 to Middleman et al. An embolic filter can be made using nitinol as shown in, for example, U.S. Pat. No. 6,179,859 to Bates et al. Also, implantable stents have been made from nitinol as shown in, for example, U.S. Pat. No. 6,059,810 to Brown; U.S. Pat. No. 6,086,610 to Duerig. A guide wire can be made from nitinol, such as that shown in U.S. Pat. No. 5,341,818 to Abrams. Nitinol is also suitable for the construction of a cardiac harness for treating congestive heart failure as seen in, for example, U.S. Pat. No. 6,595,912 to Lau. The contents of each of these disclosures are fully incorporated herein by reference.

It is understood that all nitinol alloys exhibit both superelasticity and the shape memory effect. To maximize the benefits of each, the industry has developed processing techniques to control these characteristics. Those processing techniques include changing the composition of nickel and titanium, alloying the nickel-titanium with other elements, heat treating the alloy, and mechanical processing of the alloy. For instance, U.S. Pat. No. 4,310,354 to Fountain discloses processes for producing a shape memory nitinol alloy having a desired transition temperature. U.S. Pat. No. 6,106,642 to DiCarlo discloses a process for improving ductility of nitinol. U.S. Pat. No. 5,843,244 to Pelton discloses cold working and annealing a nitinol alloy to lower the A_{f} temperature. United States Publication No. US 2003/0120181A1, published Jun. 26, 2003, is directed to work-hardened pseudoelastic guide wires. U.S. Pat. No. 4,881,981 to Thoma et al. is directed to a process for adjusting the physical and mechanical properties of a shape memory alloy member by increasing the internal stress level of the alloy by cold work and heat treatment.

One characteristic of nitinol that has not been greatly addressed is the cyclic fatigue life. In many devices, especially in medical applications, that undergo cyclic forces, fatigue life is an important consideration. There have been papers delivered on this topic such as W. Harrison, Z. Lin, “The Study of Nitinol Bending Fatigue,” pp. 391-396; M. Reinoehl, et al., “The Influence of Melt Practice on Final Fatigue Properties of Superelastic NiTi Wires,” pp. 397-403; C. Kugler, et al., “Non-Zero Mean Fatigue Test Protocol for NiTi,” pp. 409-417; D. Tolomeo, et al., “Cyclic Properties of Superelastic Nitinol: Design Implications,” all published by SMST-2000 Conference Proceedings, The International Organization Of Shape Memory And Superelastic Technology (2001). There is, however, still a need for developing a nitinol alloy that has improved fatigue life especially suitable for medical device applications.

### SUMMARY OF THE INVENTION

The present invention is generally directed to a high fatigue life metal wire, ribbon, sheet, or tubing, and processes to create such forms. In one embodiment, the high fatigue life metal wire, ribbon, sheet, or tubing comprises a core made from a binary, nickel-titanium, superelastic alloy in an ingot state having a composition of approximately 54.5 to 57.0 weight percent nickel with a balance of titanium and trace elements. The nickel-titanium alloy preferably has an ingot A_{f} temperature of approximately −15° C.±25° C.; and wherein the metal wire, ribbon, sheet, or tubing has undergone at least one cold work and anneal cycle with a final cold work of less than approximately 30% after a full anneal.

In a preferred embodiment, the metal wire, ribbon, sheet, or tubing has an ultimate tensile strength (UTS) of greater than or equal to approximately 150 ksi with an elongation at failure of greater than or equal to approximately 15%. The ultimate tensile strength and elongation specified are as measured at a temperature of approximately 23° C.±2° C. at a strain rate of approximately 0.001/sec.

The trace elements in the nickel-titanium alloy in the ingot state preferably include approximately less than or equal to 0.300 wt. % (3000 ppm) iron, less than or equal to 0.050 wt. % (500 ppm) copper, less than or equal to 0.050 wt. % (500 ppm) oxygen, less than or equal to 0.035 wt. % (350 ppm) carbon, and less than or equal to 0.003 wt. % (30 ppm) hydrogen.

Furthermore, it is preferable that any other single trace element is less than 0.1 wt. % of the alloy. Total trace elements should be less than approximately 0.4 wt. %.

Further, the cold-drawn nitinol wire, ribbon, sheet or tubing is preferably heat treated between 450-500° C. and preferably has a final A_{f} temperature between 26° C. and 36° C. as measured by bend and free recovery (“BFR”) or Differential Scanning Calorimetry (DSC).

In various alternative embodiments, the metal wire has a diameter of approximately 0.0050 inch to 0.020 inch. The wire may have a round or polygonal cross-sectional shape as with a ribbon. In accordance with the present invention, the high fatigue metal wire in a heat treated condition has a fatigue life greater than approximately 22,760 mean cycles to failure at a cyclic strain level of −0.90% to +0.90% at 37° C. as measured using a rotational beam test.

The present invention high fatigue life nitinol is preferably processed from an ingot of the composition specified above. The ingot is cold reduced or cold worked and annealed repeatedly to preferably a wire, ribbon, sheet, or tubing form. The nitinol is then cold worked through wire drawing, tube drawing, rolling, or like processes with interspersed anneal cycles for stress relief. As mentioned earlier, the final, after full anneal, cold working step is preferably limited to less than approximately 30% reduction in cross-sectional area to achieve the desired long fatigue life. In contrast, conventional processing of nitinol typically involves cold work at 35% or more.

The present invention in one embodiment limits the amount of the final cold work which, as confirmed through empirical observations, extends the fatigue life of the metal wire. The wire surface can be optionally electropolished to further improve the fatigue life. In a wire size of approximately 0.013 inch in diameter, for example, the wire fatigue life in a heat treated condition has greater than approximately 22,760 mean cycles to failure under a rotational beam test where the tested wire is subjected to an alternating strain of ±0.90% at 37° C. By comparison, standard nitinol wires in the same size and the same heat treatment condition failed under the same test at about 16,560 cycles. Based on this data, the present invention wire represents about a 37% improvement in fatigue resistance. The present invention nitinol therefore has a dramatically improved fatigue life which is highly sought after in many applications where cyclic stress or strain is present.

From empirical observations, it was determined that the ultimate tensile strength (UTS) and elongation to failure influenced the wire's fatigue resistance. Further, the amount of cold work applied to the wire during the drawing process also has an effect on the fatigue resistance. By controlling these parameters, the present invention produces a wire, ribbon, sheet or tubing having significantly improved fatigue life particularly suitable for medical device applications.

One medical device that may be constructed from the improved nitinol wire, ribbon, sheet, or tubing is a cardiac harness. The details of a cardiac harness can be found in U.S. Pat. No. 7,097,613 to Lau et al., assigned to the assignee of the present application, the contents of which are fully incorporated herein by reference. Cardiac harnesses experience a high number of stress cycles during the heart's systole and diastole, making the harness susceptible to the deleterious effects of fatigue. The process of selecting and implanting a cardiac harness on a patient is relatively invasive, and therefore it is not desirable to revisit the process because of a failure of the cardiac harness. Therefore, an improved cardiac harness that is less susceptible to fatigue effects would greatly benefit the art. The present invention includes a cardiac harness that is made from the improved, fatigue resistant nitinol and a method for using same.

It has been further discovered that a cardiac harness or other medical device exhibits other benefits from using the nitinol of the present invention in addition to improved fatigue resistance. The nitinol has been found to demonstrate improved strain characteristics that allow the cardiac harness or other medical device to operate under higher stretch percentages than heretofore. For example, prior cardiac harnesses were typically designed for a twenty-five percent stretch, with maximum of forty-five percent stretch, corresponding to a maximum mean strain in the nitinol wire of less than one percent. It was believed that maintaining the strain of the nitinol wire below one percent was necessary to prevent unwanted fatigue effects for the life expectancy of the cardiac harness. However, operating a cardiac harness in the 25%-45% stretch range lead to a very limited range of heart sizes and single cardiac harness could accommodate. This necessitated that a manufacture provide a wide array of cardiac harness sizes, and prevented a practitioner from accurately controlling the desired treatment on the patient (such as epicardial pressure).

The present invention, including a cardiac harness made from the improved nitinol, can experience a mean strain of one to three percent for a number of stress cycles that equate with a life expectancy of a cardiac harness without fatigue failure. This characteristic of the nitinol allows a cardiac harness to operate at higher stretch ranges, up to 150% and higher, without fatigue failure for the life expectancy of the cardiac harness. Operating a cardiac harness at stretch ranges higher than prior stretch ranges yields a more constant applied epicardial pressure, which translates into a more predictable epicardial pressure and less variance with the size of the cardiac harness. Less variance with size means the manufacturer can offer fewer models, and the models offered will have a more predictable effect on the patient's heart. It has even been found that a cardiac harness of the present invention, operating a high stretch ranges, can exhibit a negative slope in the stretch versus epicardial pressure graph. In other words, the cardiac harness operating in the range, e.g., 150%, may offer a “systolic kick” that actually helps to squeeze the heart during systole to a greater degree than the energy used to expand the heart during diastole. This “systolic kick” can be a significant assistance to patients with a weaker or damaged heart.

### BRIEF DESCRIPTION OF THE DRAWINGS

FIG. 1 is a graph of the effect on mean cycles to failure as a function of the ultimate tensile strength of a cold-drawn wire.

FIG. 2 is a graph of the mean cycles to failure as a function of percent elongation of a cold-drawn wire.

FIG. 3 is a graph of the effect on mean cycles to failure based on the upper plateau stress of a heat treated wire.

FIG. 4 is a graph of the effect on mean cycles to failure based on percent elongation of a heat treated wire.

FIG. 5 is a graph of the effect on mean cycles to failure based on ultimate tensile strength of a heat treated wire.

FIG. 6 is a perspective view of a cardiac harness made from a matrix of wires having high fatigue life in accordance with the present invention.

FIG. 7 is a graph of epicardial pressure against implant mean stretch for a cardiac harness comprising the present invention.

FIG. 8 is an enlarged illustration of an undeformed and deformed waveform of the present invention.

### DETAILED DESCRIPTION OF THE PREFERRED EMBODIMENTS

The present invention in various embodiments is directed to a wire, ribbon, sheet, tubing, or like structure made of superelastic nickel-titanium alloys having improved fatigue life and processes for creating such structures. Nickel-titanium alloys, also known as nitinol, have a variety of characteristics and behaviors based on processing conditions and composition. Products made from nitinol alloys nevertheless typically undergo a common series of processing steps.

For example, to produce commonly found structures such as wire, ribbon, tubing, or sheet, nickel and titanium charges are melted together to form an alloy ingot in a vacuum or inert atmosphere. Specifically, the constituent components are placed in a crucible, then induction heated or electrical arc heated in a vacuum induction melting (VIM) process or vacuum arc remelting (VAR) process, respectively. The nitinol ingot after VIM or VAR processing has the general composition of nickel to titanium as well as trace elements of carbon, oxygen, iron, and other impurities. After the melting process, the nitinol ingot has little ductility, and accordingly, it is preferable to hot work the ingot to achieve a microstructure that exhibits better workability.

To move the material closer to the desired mechanical and physical properties, the nitinol ingot undergoes a series of cold working steps. Typically, the nitinol receives cold working in the range of 40 to 50% at each step, and is also annealed at about 600 to 800° C. for stress release after each cold work step. The interspersed anneal cycles minimize work hardening of the nitinol caused by the repeated cold work. The cold working is typically performed by cold drawing for wires and ribbons through a series of dies; cold rolling for sheet stock; and tube drawing with an internal mandrel for tubes. To obtain the desired superelastic or shape memory properties, the nitinol alloy is usually heat treated after the last cold work step at about 450 to 550° C. Further details regarding conventional nitinol processing and fabrication are disclosed in, for example, Scott M. Russell, “Nitinol Melting and Fabrication,” SMST-2000 Conference Proceedings, pp. 1-9 (2001), whose entire contents are hereby incorporated by reference. At this stage, the nitinol wire or ribbon, sheet stock, or tube has been transformed from raw materials into a standardized, nearly finished condition for consumption in the industry.

As explained earlier, the transformation temperature of the nitinol separates the austenitic phase from the martensitic phase. Typically, the transition temperature is measured by the austenite finish (A_{f}) temperature, which indicates the completion of the phase transformation from martensite to austenite during heating. The alloy transformation temperatures are determined by, among other factors, the ratio of nickel and titanium in the alloy. To be sure, the transformation temperatures are extremely sensitive to very small changes in the Ni—Ti composition. As a result, the presence of impurities or trace elements aside from nickel and titanium might unexpectedly change the transformation temperature of the alloy.

The A_{f} temperature is commonly used as a metric in defining the characteristic of a nitinol device since it defines when the nitinol is completely in the austenitic phase. The A_{f} temperature is usually measured by a technique called Differential Scanning Calorimetry (DSC) or by a “bend and free recovery” (“BFR”) technique. The DSC technique detects the heat released and absorbed during the martensitic (exothermic) and austenitic (endothermic) transformations, respectively, and thus produces data indicating A_{f} temperature. The bend and free recovery technique requires cooling the nitinol sample to a low temperature so that it is in the martensitic phase, bending the sample to a prescribed strain (typically 2% to 3%), and observing the temperature at which the sample returns to its original shape in the austenitic phase when heated, thus indicating the A_{f} temperature. The nitinol ingot can be Vacuum Induction Melting (VIM) melt processed in a graphite crucible to reduce oxide formation, which results in a nitinol that has a slightly higher carbon content, 0.0365% by weight compared to 0.003% of other typical nitinol sources). This and other processing steps are taken to reduce impurities in the resultant material. The result is an ingot which, when drawn down to a wire diameter typically used in medical devices (e.g. 0.003″-0.250″), results in smaller typical inclusions in the range of two microns, and stringers that are one tenth the typical length. The ingot typically has a greater number of these smaller inclusions than for typical nitinol.

Another metric for working with nitinol is the “ingot transition temperature.” This is commonly defined as the A_{f} temperature after a “full anneal” of the alloy. A full anneal implies that the alloy has been completely stress relieved, typically at about 750° C. for 5 to 10 minutes. The ingot transition temperature is usually measured by use of a DSC. The ingot transition temperature is indicative of the chemical composition of the alloy in the ingot state. As is known in the art, heat treatment and cold work can change the transition temperature of the alloy. For a metric that reflects the processing received by the alloy, the “final A_{f} temperature” is used. The final A_{f} temperature is determined by using the DSC or BFR test on the alloy after it has been shape set to its remembered shape.

The present invention in various embodiments is directed to a high fatigue life metal wire, ribbon, tubing or sheet stock. In one preferred embodiment, the composition of the nitinol alloy in the ingot state includes about 55.8 weight percent nickel and about 44.2 weight percent titanium. In various alternative embodiments, the nickel composition may range from about 54.5 to 57.0 wt. % and everything therebetween, with the balance titanium (i.e., 45.5 to 43.0 wt. % and everything therebetween). Trace elements or impurities may be present but are preferably limited to the following approximations: iron ≦0.300 (3000 ppm); copper ≦0.050 (500 ppm); oxygen ≦0.050 (500 ppm); copper ≦0.035 (350 ppm); and hydrogen ≦0.003 (30 ppm). Any other single trace element should preferably be ≦0.1 weight percent. The total amount of trace elements present should be ≦0.4 weight percent. Furthermore, the ingot transformation temperature (A_{f}) as measured in the fully-annealed condition by the DSC technique should preferably be about −15° C.±25° C.

Once the composition and transformation temperatures for the ingot are set as above, the ingot undergoes a sequence of cold working and anneal cycles to reduce the ingot into preferably a wire, ribbon, tubing, or sheet of a desired cross-sectional area through the processing steps explained above.

In a preferred embodiment of the present invention, the “final” cold work or cold drawing step of the wire, ribbon, tubing or sheet stock is limited to less than approximately 30%, more preferably in the range of about 27%±3%. The “final” cold work or cold drawing step refers to the step immediately after a full anneal of the nitinol part in which the nitinol part undergoes a cold reduction or deformation changing the nitinol part into the desired final dimensions.

A further preferred embodiment of the present invention contemplates that the finished wire, ribbon, tubing, or sheet stock possess an ultimate tensile strength of approximately ≧150 ksi with an elongation at failure of approximately ≧15% as measured at a temperature of about 23° C.±2° C. at an approximate strain rate of 0.001 per second. More preferably, the UTS may be ≦190 ksi and ≧150 ksi including everything therebetween, while the elongation at failure may be ≦40% and ≧15% including everything therebetween. These parameters are again achieved through the sequence of cold work and anneal cycles mentioned above.

For high-cycle alternating strains, the result is very fatigue resistant nitinol wire when it is finally drawn down from the specially processed ingot. Preliminary testing (rotary beam testing) has demonstrated that nitinol wire from the specially processed ingot was up to two times as fatigue resistant as typically used nitinol. In this test, the heat treated wire specimen with an A_{f} temperature of 32±3° C. is gripped at the opposite ends where one end is motor driven and where both gripped ends are parallel and co-planar. The entire specimen is held within a vertical plane with the motor-driven end rotating to create alternating compressive and tensile strain in the specimen. The alternating strain ranged from about −0.90% to +0.90%. The specimen was also immersed in a water bath at 37° C. to approximate human body temperature. Being above the A_{f} temperature of the wire, the ambient temperature also places the superelastic nitinol specimen in the austenitic phase. The motor-driven end rotated the specimen at a rate of 3,600 cycles per minute. In this test, the standard nitinol wire with a cold work of 40%±5% failed at an average of about 16,560 cycles; one embodiment of the present invention nitinol wire failed at about 22,760 cycles, which is an improvement of 37% in fatigue life. In the above testing, a standard nitinol wire was used for comparison against one embodiment of the present invention. Both specimens were 0.013 inch diameter wire, with the same shape-setting heat treatment, having a nominal composition of 55.8 wt. % nickel and 44.2 wt. % titanium. Both have a total trace element composition of <0.4 wt. %. The following are the differences between the standard nitinol wire versus the present invention nitinol wire. Standard nitinol wire: 40%±5% final cold work; ingot A_{f} temperature −15 to +15° C.; UTS ≧190 ksi, elongation at failure ≧6% at room temperature. Tested embodiment of invention: 27%±3% final cold work; ingot A_{f} temperature −40 to +10° C.; UTS ≧150 ksi, elongation at failure ≧15% at room temperature.

Further nitinol wire fatigue testing looked at wire that had been formed, heat treated and polished to the final device configuration of FIG. 6. The in-vitro testing simulated anticipated use conditions (high cycle fatigue with a mean and alternating component) showed more dramatic gains in fatigue resistance. This testing was conducted to form an S-N Curve (also known as a Wohler curve), a test where the wire is strained at different levels and the number of cycles to fatigue is tracked. By developing fatigue failure data at different strain levels the goal is to find the endurance limit where below a certain strain limit the device would have endless life. The mechanical motion of the S-N test was a repeating axial stretching of the device (see FIG. 8), where waveform 300 represents an undeformed (0% stretch) configuration and waveform 400 represents a 100% stretched configuration. The test simulates the stretching and contraction of a harness on the heart. The S-N test was conducted up to 100 million cycles after which the test was stopped assuming the device would not fail beyond that time. The mean strain level was changed during the test with the alternating strain staying a constant 13% stretch of the standard coupon length. The results of the SN test showed that the nitinol that was specially treated could not be induced to fail even at mean stretches as high as 187%, whereas the S-N test of the standard nitinol appeared to have a strain endurance limit at 112% mean stretch. Thus the specially treated nitinol has a much higher endurance limit than the standard nitinol.

One difference between the standard wire versus the present invention wire is the amount of final cold work, where the amount of the final cold work step in the present invention wire is much lower. The expression “final cold work” as defined earlier is intended to mean the last cold work step bringing the part into its final dimensions, after a full anneal, and before the shape setting step where the shape memory is imparted into the alloy. From the test data, it is preferred that the final amount of area reduction by the cold working—such as wire drawing—is limited to less than 30%, and more preferably in the range of 27%±3% in order to help achieve the desired long fatigue life.

A coupon fatigue test was also used. The coupon test involves gripping the opposite ends of the specimen, which has a two-dimensional configuration imparted by the shape-setting treatment. The motorized test fixture then uniaxially tensions and releases the tension on the specimen. This is performed in a saline bath maintained at 37° C. The cycle rate of the test fixture is 15 cycles per second. At an aggressive loading condition of 80% to 120% stretch ratio based on the initial gauge length of the test specimen and corresponding to strain levels of approximately 0.9% to 1.4%. Under this test, the standard nitinol failed after an average of 7.3 hours (approximately 32000 cycles). Specimens of the present invention survived over 12 months (approximately 38 million cycles) and up to 15.3 months (approximately 48 million cycles) without failure. These empirical observations further confirmed the improved fatigue life of the present invention alloy and processing steps.

In nitinol it is believed that the build up of dislocations can be reduced by stress induced martensite formation in the material under stress. Past research has shown that martensite transformation is initiated from nucleation sites. Inclusions can act as initiation sites to start the martensitic transformation. Having lots of small inclusions can lead to a more heterogeneous microstructure which can in turn lead to a more homogeneous strain across the area of the strained wire. A more homogeneous transformation strain will favor a greater stability against fatigue. By using the small inclusion nitinol wire, the peak strain that the wire experiences can be reduced, which in turn would reduce the probability of dislocation build up that leads to crack initiation, thereby improving fatigue life. A more homogeneous material strain can also slow the rate of crack propagation, thus improving fatigue life. These improved affects on fatigue for the small inclusion wire are different than the fatigue seen with large inclusion wire where the fatigue life is determined by inclusion initiated phenomena.

By changing fatigue fracture initiation mechanisms and lowering the rate of fracture initiation, the small inclusion material allows a device constructed therefrom to be used in a high mean stretch configuration. With the present invention, the capability of a high mean stretch results in a condition where the material strain is on the plateau of the hysteresis curve. This allows the material to stay in a pure stress induced martensite crystal structure through the duration of the stress cycle allowing for better fatigue life

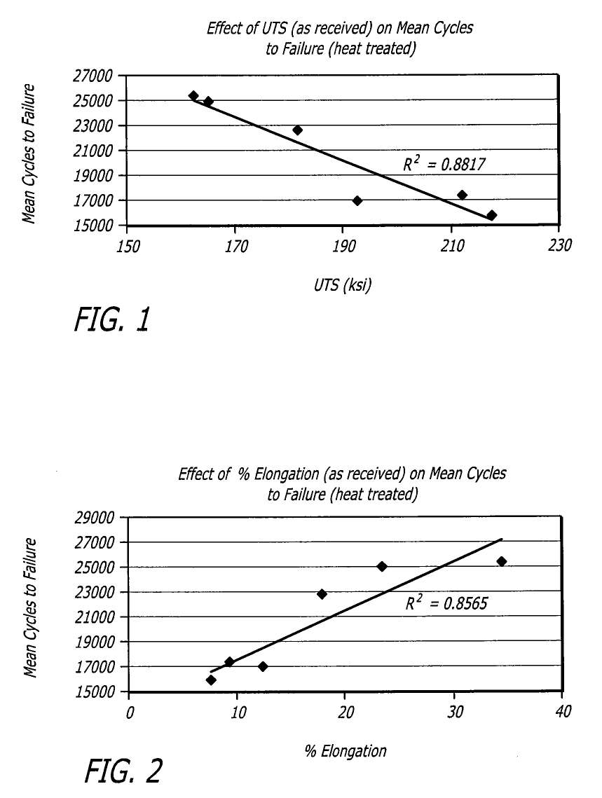

FIGS. 1 and 2 are plots of test data generated by 0.013 inch diameter nitinol wire made in accordance with the present invention. A rotational beam fatigue test was applied to these specimens using a 0% mean strain and an alternating strain of ±0.90%. FIG. 1 shows the influence of the ultimate tensile strength (UTS) on the mean cycles to failure. FIG. 2 is a plot showing the influence of percent elongation on the mean cycles to failure in the wire specimens. Note that the fatigue test was conducted after the shape-setting heat treatment on the specimens, but the UTS and elongation to fatigue were measured on the wire specimen in the as-drawn condition.

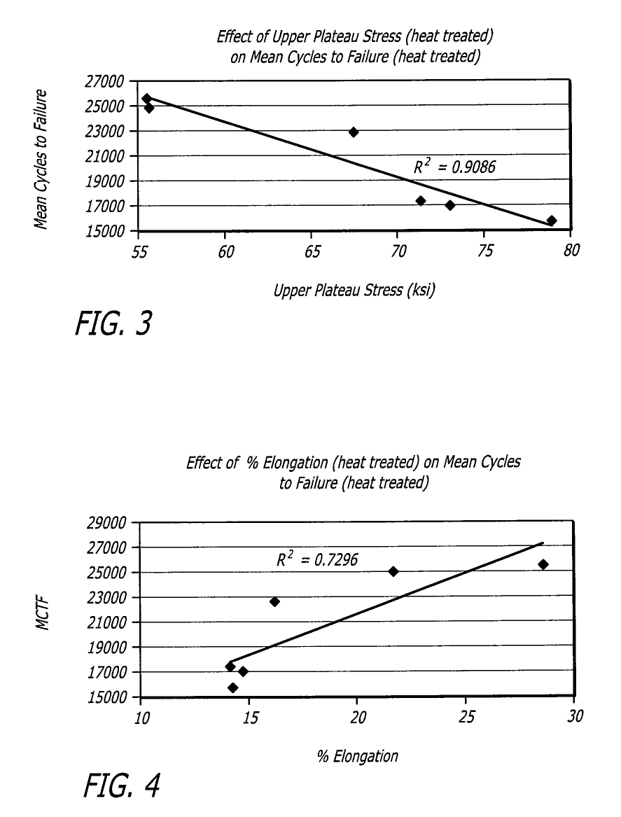

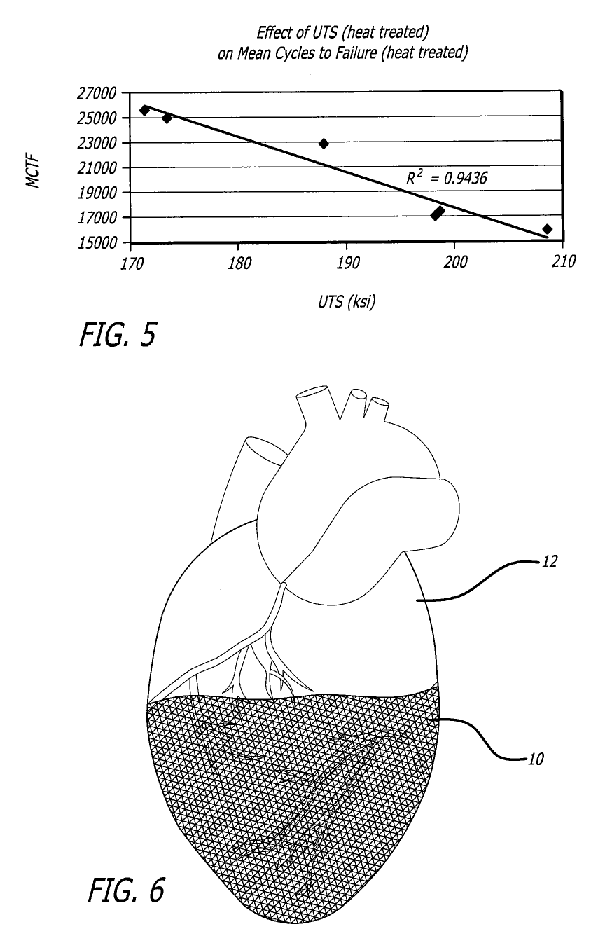

FIGS. 3-5 are plots of the specimens described above under the same rotary beam fatigue test, but all properties were measured after the shape-setting heat treatment on the specimens. In FIG. 3, the upper plateau stress (from the superelastic nitinol stress-strain “flag” curve) of the superelastic nitinol alloy is plotted against the mean cycles to failure. In FIG. 4, the percent elongation is plotted against the mean cycles to failure while in FIG. 5, the ultimate tensile strength is plotted against the mean cycles to failure.

Based on the foregoing plots and other empirical observations, it was determined that in order to achieve an improved fatigue life, it is desirable to limit the final cold work step after the precursor cycles of cold drawing and annealing, to less than approximately 30%, and more preferably in the range of 27%±3%, and even down to 24%. Ideally, the ultimate tensile strength should preferably be set at ≧150 ksi with an elongation at failure preferably set at ≧15%.

The tested specimens in the described rotary beam fatigue test were not polished after the shape setting heat treatment. Therefore, they exhibited a blue oxide surface.

The present invention nitinol wire, ribbon, tubing or sheet stock can be shape set to the desired shape through processes known in the art. This is usually accomplished by manipulating the nitinol wire, ribbon, tubing, or sheet into a fixture duplicating the remembered shape. The nitinol wire, ribbon, tubing or sheet is heated to well above the alloy's martensite deformation temperature (Md). For a wire, ribbon, tubing, or sheet, the shape set temperature is typically in the range of 250-600° C.; the heating occurs for an average of a few minutes up to an hour, with longer times for lower temperatures and vice versa.

The cold-drawn nitinol wire embodiment is preferably heat treated between 450-500° C. and preferably has a final A_{f} temperature between 26° C. and 36° C. as measured by the DSC or BFR technique.

The blue oxide surface formed from the shape setting heat treatment can optionally be removed by electropolishing. This further improves fatigue resistance. Moreover, the final A_{f} temperature of the formed wire can optimally be adjusted by the shape setting heat treatment without deviation from the scope of the present invention.

In one preferred application, the present invention high fatigue life wire or ribbon can be constructed into a matrix or wire mesh for use as a cardiac harness for treating congestive heart failure, shown in FIG. 6. As referenced above, details of a cardiac harness can be found for example in U.S. Pat. No. 7,097,613 to Lau et al. The wires or ribbons may be interlocked, interwoven, or otherwise joined together forming a sleeve. If a sheet or tube of the present invention high fatigue life nitinol is chosen as the foundation, then it can be laser cut, electro-discharge machined, chemically etched, or likewise cut to create a pattern of openings to form a matrix that is then shaped into a sleeve also suitable for use as a cardiac harness.

In a patient with congestive heart failure, the diseased myocardium begins to remodel which typically manifests in the heart enlarging into a more spherical shape. One type of treatment is to implant an external elastic support or constraining sleeve for the myocardium. Such a constraining sleeve, called a cardiac harness 10, is seen in FIG. 6. In this embodiment, the cardiac harness 10 surrounds both ventricles, from apex to base of the heart 12. As the ventricle dilates in congestive heart failure, outward radial pressure is applied to the cardiac harness 10; conversely, the cardiac harness applies a constraining pressure on the heart.

More important is the systole and diastole contraction and relaxation of the heart which apply repeated cyclical pressure on the cardiac harness 10. Due to this cyclic stress, the cardiac harness should exhibit a relatively high fatigue life after implantation in the patient. Therefore, the wires forming the cardiac harness 10 are made from superelastic nitinol in accordance with the present invention embodiments and are in the austenitic phase at body temperature when no load is applied and the alloy is stress-free. When placed over the heart as shown in FIG. 6, the contact pressure (hereafter “epicardial pressure” or “epicardial contact pressure”) between the harness 10 and heart 12 may create stress-induced martensite (SIM) in the material. Depending on the stress-strain “flag” curve of the superelastic nitinol alloy, the actual stress encountered by the nitinol wire may fall on a stress plateau or may be sufficiently low to fall in the linear stress-strain range. In any event, the present invention high fatigue life wire minimizes the possibility under such conditions of a fracture or fatigue failure in the harness.

Cardiac harnesses are currently manufactured in multiple sizes to accommodate a range of heart circumferences and lengths. Prior art cardiac harnesses targeted an operational expansion or “stretch” range of 25%-45% relative to their unstretched circumference, regardless of the size of the underlying heart. However, according to LaPlace's Law (equation I) the resultant therapy in the form of epicardial contact pressure imposed by the implant will diminish with increasing heart circumference:

P=F₁(S)/R_{H}·W₁ (I)

where R_{H} is the equivalent heart radius, F₁(S) is the circumferential hoop force generated by a functional width of the implant W₁ (for example, a ring of the implant), at a specified stretch S. Using F₁ (S) as determined from a least-squared regression fit to empirical compliance data collected from force-vs-length Instron testing of the fundamental teardrop structure of a class of cardiac harnesses, LaPlace's Law can be used to estimate the theoretical epicardial contract pressures across a range of implant sizes.

Based on the LaPlace equation (I) above, the target stretch range of 25%-45% predicts that a ten spine implant would theoretically be able to generate 0.72-1.03 mmHg of epicardial contact pressure, while a fifteen spine implant would only be able to generate 0.48-0.69 mmHg of epicardial pressure. Therefore, assuming contact pressure provides the primary mode of implant therapy, patients with larger hearts (and thus who consequently receive larger implants) would be at a therapeutic disadvantage.

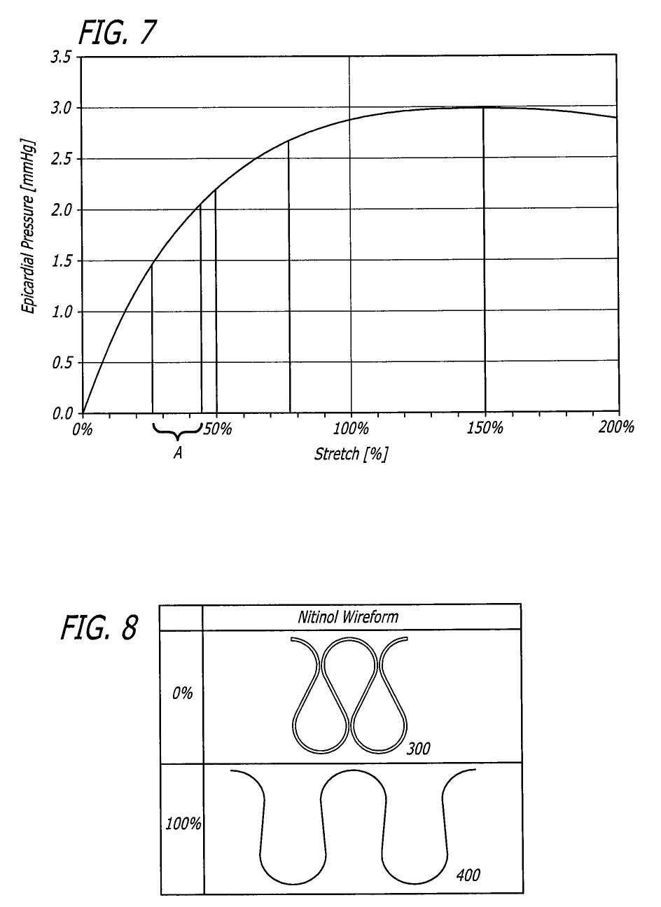

Fatigue and failure criteria are largely responsible for the limitation that prior art cardiac harnesses were limited to a mean stretch of approximately 25%-45%. This is consistent with a mean strain of approximately one percent on the underlying materials used to construct the harness. One percent mean strain has been a ceiling above which manufactures have refrained from exceeding due to the deleterious effects that a higher mean strain has on fatigue. Operating at or below one percent mean strain of the materials limits the absolute stretch of the harness to the twenty-five to forty-five percent (designated “A” in FIG. 7). In FIG. 7, for a cardiac harness 10 made of the superelastic nitinol, the mean stretch percentage of the diameter of the harness is plotted against the applied epicardial pressure imposed on the heart by the harness as the harness expands from its relaxed state. At a stretch range of 25%-45%, there is a high epicardial pressure differential for relatively small changes in the mean stretch of the cardiac harness. This phenomenon is seen even in the cardiac harness 10 made from the superelastic nitinol, the subject of FIG. 7, but is even more pronounced in prior art cardiac harnesses.

From FIG. 7, it can be seen that a cardiac harness will impose on a heart about 1.4 mmHg of pressure for a stretch of twenty-five percent, and 2.1 mmHg of pressure at forty-five percent. An implant sized for this stretch range will experience approximately a fifty percent increase in pressure for only twenty percent absolute change in the stretch of the harness. This characteristic requires that the sizing of the implant to be very precise in order to achieve the desired cardiac pressure, and that even with a precise sizing the fluctuation in applied pressure will be large. This also requires that the manufacturer make and carry a large variety of harness sizes.

The material of the superelastic nitinol of the present invention, however, can safely operate at a higher mean strain than prior art cardiac harness materials. Testing has shown that a mean strain of up to three percent or more can be safely utilized in sizing and implementing a cardiac harness 10 without risking fatigue failure. Using a mean strain in the superelastic nitinol of up to and even exceeding three percent, as FIG. 7 illustrates, the epicardial pressure versus stretch curve for a cardiac harness begins to flatten out at about fifty percent stretch of the implant, and to an even greater extent at seventy-five percent stretch. Beyond this mean stretch, the implant experiences only small changes in epicardial pressure even for relatively high changes in absolute stretches. Sizing new cardiac harnesses 10 to operate at mean strains of three percent or more, such that the implant operates within a range of mean stretches in the range of fifty percent to one hundred fifty percent, and more favorably between seventy-five percent and one hundred fifty-percent, results in a harness that is very robust to sizing and achieves a more constant epicardial pressure for all operating conditions. In the stretch range of between seventy-five percent and one hundred fifty percent, a change in epicardial pressure is less than 0.5 mmHg. Accordingly, if the heart begins to reverse remodel due to the presence of the harness, the applied force of the cardiac harness 10 will remain constant if sized to operate with the flattened portion of the curve of FIG. 7.

Conventional fatigue theory suggests that the cardiac harness 10 when operating under the higher strains would be unsuitable for the present application, where failure would be expected anywhere between two hundred to two million cycles. This results in a factor of safety well below acceptable standards. However, testing has established that the superelastic nitinol's fatigue characteristics operating in the high mean strain conditions (>3%) still resulted in usable lifetimes with favorable factors of safety.

TABLE 1
Epicardial Contact Pressure [mmHg]

Number of Spines in Cardiac Harness

| % Stretch | 10 | 11 | 12 | 13 | 14 | 15 |
|---|---|---|---|---|---|---|
| 0% | 0 | 0 | 0 | 0 | 0 | 0 |
| 5% | 0.2 | 0.18 | 0.16 | 0.15 | 0.14 | 0.13 |
| 10% | 0.36 | 0.33 | 0.30 | 0.28 | 0.26 | 0.24 |
| 15% | 0.50 | 0.45 | 0.42 | 0.38 | 0.36 | 0.33 |
| 20% | 0.62 | 0.56 | 0.52 | 0.48 | 0.44 | 0.41 |
| 25% | 0.72 | 0.66 | 0.60 | 0.56 | 0.52 | 0.48 |
| 30% | 0.82 | 0.74 | 0.68 | 0.63 | 0.58 | 0.54 |
| 35% | 0.90 | 0.81 | 0.75 | 0.69 | 0.64 | 0.60 |
| 40% | 0.97 | 0.88 | 0.81 | 0.74 | 0.69 | 0.65 |
| 45% | 1.03 | 0.94 | 0.86 | 0.79 | 0.74 | 0.69 |
| 50% | 1.09 | 0.99 | 0.91 | 0.84 | 0.78 | 0.73 |
| 55% | 1.14 | 1.04 | 0.95 | 0.88 | 0.82 | 0.76 |
| 60% | 1.19 | 1.08 | 0.99 | 0.92 | 0.85 | 0.79 |
| 65% | 1.24 | 1.12 | 1.03 | 0.95 | 0.88 | 0.82 |
| 70% | 1.28 | 1.16 | 1.06 | 0.98 | 0.91 | 0.85 |
| 75% | 1.31 | 1.19 | 1.10 | 1.01 | 0.94 | 0.88 |
| 80% | 1.35 | 1.23 | 1.12 | 1.04 | 0.96 | 0.90 |
| 85% | 1.38 | 1.26 | 1.15 | 1.06 | 0.99 | 0.92 |
| 90% | 1.41 | 1.28 | 1.18 | 1.09 | 1.01 | 0.94 |
| 95% | 1.44 | 1.31 | 1.20 | 1.11 | 1.03 | 0.96 |
| 100% | 1.46 | 1.33 | 1.22 | 1.13 | 1.05 | 0.98 |

The present invention allows the designers of cardiac harnesses the flexibility to size implants based on a target epicardial pressure rather than a target stretch range. Table 1 illustrates how the stretch windows would extend for each implant size if the epicardial pressure were targeted at roughly 1.03 mmHg (bolded portion). Whereas the operating stretch range for a 10 spine implant would still be 25%-45% to achieve maximum target epicardial pressure of 1.03 mmHg, the upper limit upper limit operating stretch range for the 11, 12, 13, and 14 spine implants would theoretically increase to 55%, 65%, 80%, and 95%, respectively. Thus, when sized in this manner, the therapy (e.g., contact pressure) applied by the implant is equivalent across all implant sizes, regardless of the patient heart's size. This approach can also be generalized in several ways. For example, since the effective radius of the heart diminishes from base to apex, the design of the implant is tailored such that multiple sections, or even each ring, of the implant are individually optimized to achieve a target therapeutic effect such as a specified epicardial pressure.

This characteristic of the present invention simplifies the sizing process for selecting the size of medical implants such as cardiac harnesses and stents. Most measurement techniques (and especially echocardiography) have a considerable amount of error in the measurement. A product that can be used over a wider range of measurements will equate with performance that is less sensitive to measurement errors in sizing. This consideration is important as most current Nitinol medical implants are intended to be used within vessels in the form of stents and stent-grafts. For this use, proper sizing of the vessel and the implant is vital. If the implant is oversized, it may block the bloodstream or create eddies or spots where thrombosis is likely. If the implant is undersized then it may migrate downstream. Accordingly, devices with this requirement have many specifically-sized product offerings. In intralumenal applications, the large elastic plasticity range for nitinol is the primary advantage of the material, allowing it to be elastically-crushed down on a delivery system while being self-expanding upon deployment.

By utilizing a nitinol that is more fatigue resistant, a cardiac harness can handle a greater amount of strain and specifically a wireform that can handle a greater amount of stretch. This results in several advantages. For a device placed on the outside surface of an organ or structure, a device that can handle a greater amount of stretch can have therapeutic advantages. In the case of a cardiac harness, it is anticipated that the cardiac harness would stop cardiac remodeling and may allow the heart to reverse remodel. In present cardiac harnesses, with a smaller stretch range, the heart can only remodel a small amount before the device reaches its unstretched size and stops providing pressure-relieving therapy to the heart. If a device with a greater stretch range is implanted, even after the heart remodels some, the device will still stretch and provide therapy. Thus there is a greater range for the heart to continue to remodel to the point that the heart approaches a normal size.

For a device with a fairly flat compliance curve, LaPlace's law suggests that a more uniform therapy (pressure) may be applied by the implant over a greater operating range. The active portion of the compliance curve and the resultant pressure curve can be designed to be much flatter because the device can operate at a greater amount of stretch. Where this is an advantage is that with a flatter pressure curve, the curve that describes the therapy on the heart, the therapy is more consistent across a range of heart sizes. Thus therapy can be much more targeted and is less subject to variation as actually applied to the heart. This means that the therapy delivered as a heart reverse remodels or if a heart continues to get larger can remain fairly constant. For hearts that reverse remodel, an optimal amount of reverse remodeling can then be achieved. For hearts that continue to enlarge, therapy can continue to be delivered without fear of the device causing constriction of the heart.

A high fatigue life (and resultant large operating range) combined with the uniform therapy of the device may also enable the device to be used synergistically with other therapies. For example, by using a highly stretched device a cardiac harness could be implanted at the same time as an LVAD for a patient. Patients with LVADs have a tremendous amount of reverse remodeling. Normally this would create a mismatch in fit between the cardiac harness and the heart, but if a highly-stretched harness is used the harness can continue to deliver appropriate support therapy even after significant reverse remodeling. The implantation of an LVAD creates a myriad of pericardial adhesions. These adhesions may make it impossible to correctly deliver a cardiac harness over the heart. By implanting the harness at the same time as LVAD implantation the problem of these adhesions during delivery are avoided.

A device which can handle a greater amount of stretch can have functional advantages. As previously mentioned for the current wireform configuration, greater stretch allows the wireform to operate where the wire material strain is on the plateau of the hysteresis curve. This increases the fatigue life of the material and the device. A device which can handle a greater amount of stretch will physically be a device with less total volume. This is important because the device is delivered through an incision in the chest wall. The reduced volume of the device would allow for a smaller delivery system which would allow for smaller thoracic and pericardial incisions, resulting in less rib spreading and tissue trauma and faster healing with less recovery complications for the patient.

One preferred embodiment for use of the high fatigue life nitinol is a nitinol cardiac harness, preferably one that applies an average external pressure that may range from 0.5 mmHg to 5 mmHg and that would be sized to fit hearts with a base ventricular epicardial circumference of approximately 225 mm to 460 mm. This device would be capable of being stretched up to 150% or more of its original circumference upon implantation and would also survive the alternating stretch conditions applied during the cardiac cycle.

Another phenomenon is illustrated in FIG. 7 at the portion of the curve to the right of the apex (approximately 150% stretch). It can be seen that the cardiac harness 10 experiences a negative slope where the mean stretch percentage is higher than approximately one hundred fifty. This is the result of epicardial pressure (or hoop stress) being dependent on the radius of the implant at a given stretch. A cardiac harness operating in this range would experience a potentially beneficial boost in pressure during systole. That is, as the heart contracts the cardiac harness applies an increasing pressure or “systolic kick” to reduce the amount of work that the heart performs during contraction. For this application, “systolic kick” means that as the cardiac harness contracts from a more stretched configuration to a less stretched configuration, the applied epicardial pressure increases for at least a portion of the contraction. This systolic kick could improve heart function and enhance the performance of a weakened heart, which in turn could extend the life of a patient.

Another medical application of the high fatigue life wire is in the area of implantable stents. A stent implanted in a vessel behind the knee would certainly encounter cyclic stresses and strains and long fatigue life becomes an important consideration. Stents also can experience cyclic loading, and a stent that is made of the superelastic nitinol and operated at higher mean stresses such as those discussed above with respect to cardiac harnesses can benefit from the discoveries of the present invention. Other applications include, for example, eyeglass frames, cell-phone or radio antennas. Such applications expose the wire to cyclic stresses and strains, and a high fatigue life is unquestionably a valuable engineering asset.

Various modifications may be made to the present invention without departing from the scope thereof. Although individual features of embodiments of the invention may be shown in some of the drawings and not in others, those skilled in the art will recognize that individual features of one embodiment of the invention can be combined with any or all of the features of another embodiment.

We claim:

1. A method for using a nickel-titanium alloyed member having a composition of approximately 54.5 to 57.0 wt. % nickel comprising:
forming the member into a component of a system where the component is subject to repeated loading conditions during operation; and
placing the member into the system where the member experiences a mean strain of between one percent and approximately three percent for the life of the member.

2. The method for using a nickel-titanium alloyed member of claim 1, wherein the system is a human body.

3. The method for using a nickel-titanium alloyed member of claim 2, wherein the component is a cardiac harness.

4. The method for using a nickel-titanium alloyed member of claim 3, wherein the cardiac harness is sized to operate within a mean stretch percentage of between fifty percent and one hundred fifty percent.

5. The method for using a nickel-titanium alloyed member of claim 4, wherein the cardiac harness is sized to operate within a mean stretch percentage of between seventy-five percent and one hundred fifty percent.

6. The method for using a nickel-titanium alloyed member of claim 3, wherein a change in epicardial pressure applied to a heart by the cardiac harness is less than 1.0 mmHg for an operating range of the cardiac harness.

7. The method for using a nickel-titanium alloyed member of claim 3, wherein the cardiac harness is sized for operating in a range above one hundred fifty percent mean stretch for at least a portion of an operating cycle of the member.

8. The method for using a nickel-titanium alloyed member of claim 3, wherein the cardiac harness is sized to produce a systolic kick during contraction of the heart.

9. The method for using a nickel-titanium alloyed member of claim 1, wherein the member is a wire having a diameter of between 0.005 and 0.020° inches.

10. The method for using a nickel-titanium alloyed member of claim 3, wherein the cardiac harness comprises a series of segments, and each segment is optimized to achieve a target therapeutic effect.

11. The method for using a nickel-titanium alloyed member of claim 10, wherein a segment comprises at least one interlocking ring.

12. The method for using a nickel-titanium alloyed member of claim 10 wherein the target therapeutic effect is epicardial contact pressure.

13. A medical device for implantation, comprising:
a sleeve having elastic compliance under expansion forces, the sleeve comprising a binary alloy of nickel and titanium where a percentage of nickel is between 54.5 and 57.0 percent;
wherein the sleeve is subject to a repeated loading condition after implantation; and
wherein the sleeve is sized to operate at a mean strain of greater than one percent.

14. The medical device for implantation of claim 13, wherein the sleeve is a cardiac harness.

15. The medical device for implantation of claim 14, wherein the cardiac harness has an unloaded condition, and the cardiac harness is sized to operate at a mean stretch percentage of greater than fifty percent above its unloaded condition.

16. The medical device for implantation of claim 15, wherein the cardiac harness is sized to operate at a mean stretch percentage of greater than seventy-five percent above its unloaded condition.

17. The medical device for implantation of claim 14, wherein the cardiac harness is sized such that it imposes a systolic kick on a heart during at least a portion of a contraction of the heart.

18. The medical device for implantation of claim 14, wherein the cardiac harness is sized such that it imposes an epicardial pressure within a range of less than 1.0 mmHg for an operating range of the cardiac harness.

19. The medical device for implantation of claim 13, wherein the sleeve is sized to operate at a mean strain of greater than three percent.

20. The medical device for implantation of claim 13, wherein the sleeve comprises a plurality of segments, and each element is optimized to achieve a target therapeutic effect.

21. The medical device for implantation of claim 20, wherein a segment comprises at least one interlocking ring.

22. The medical device for implantation of claim 20, wherein the target therapeutic effect is an epicardial contact pressure.

23. The medical device for implantation of claim 13, wherein the sleeve is a stent implant.

24. A method for selecting a cardiac harness to be applied to a heart, comprising:
determining a desired epicardial pressure to be applied by the cardiac harness on the heart; and
using a relationship between epicardial pressure and cardiac harness circumference to obtain an ideal cardiac harness size for the desired epicardial pressure.

25. The method of selecting a cardiac harness of claim 24, further comprising determining a desired epicardial pressure for a plurality of longitudinal locations along the heart, and using the relationship between epicardial pressure and cardiac harness circumference obtaining a circumferential size for a plurality of longitudinal locations along the cardiac harness.
